(19) United States
(12) Patent Application Publication
(10) Pub. No.: US 2025/0345324 A1
(43) Pub. Date: Nov. 13, 2025

(54) COMBINATION FORMULATIONS OF NALOXONE AND ATIPAMEZOLE

(71) Applicants: HEMANT NARAHAR JOSHI, PARSIPPANY, NJ (US); NEELAM SHARMA, MORRIS PLAINS, NJ (US); LAVANYA KUNDURTHY, MORRIS PLAINS, NJ (US)

(72) Inventors: Hemant NARAHAR Joshi, PARSIPPANY, NJ (US); NEELAM SHARMA, MORRIS PLAINS, NJ (US); LAVANYA KUNDURTHY, MORRIS PLAINS, NJ (US)

(73) Assignee: HEMANT NARAHAR JOSHI, PARSIPPANY, NJ (US)

(21) Appl. No.: 18/658,098

(22) Filed: May 8, 2024

(51) Int. Cl. A61K 31/485; A61K 9/00; A61K 9/06; A61K 9/20; A61K 9/46; A61K 31/4164; A61K 47/10; A61K 47/18; A61K 47/36; A61K 47/38

(52) U.S. Cl. CPC A61K 31/485; A61K 9/0007; A61K 9/0014; A61K 9/0019; A61K 9/0043; A61K 9/0056; A61K 9/06; A61K 9/2009; A61K 9/2018; A61K 9/2027; A61K 9/2054; A61K 31/4164; A61K 47/10; A61K 47/183; A61K 47/36; A61K 47/38

(57) ABSTRACT

Naloxone is commonly used to treat opioid overdose. The present invention presents a combination of naloxone with atipamezole. The invention presents various dosage forms to be administered parenterally, orally or topically. The formulations may also contain a chelating agent such as disodium EDTA. These combination formulations can be used to treat drug addicts.

### FIELD OF INVENTION

This patent application relates to the combination drug delivery systems of Naloxone and Atipamezole. Injections (immediate release and sustained release), local application (nasal and topical), and tablets (sublingual, effervescent, gelling tablet) combination formulations of these were prepared.

### BACKGROUND OF THE INVENTION

Drug addiction is a complex and challenging problem that affects individuals and communities worldwide. Overdose from opioids and certain sedative drugs is a major concern in the context of substance abuse. Illegal drug usage has been increasing every day. Drug addicts do not necessarily have low will-power. Drug addiction is a complex disease and quitting takes more than a strong will. Drug addiction is compulsive and difficult to control, despite harmful consequences. This is due to changes in the brain resulting in their ability to resist an intense urge to take drugs. Drugs cause euphoria due to flooding with dopamine. It taps into the reward system of the brain motivating the person to repeat drug usage. Long-term usage of drugs affects the following functions-learning, judgement, decision-making, stress, memory and behavior.

The human opioid system contains four opioid receptors (μOR, δOR, κOR, and NOPR) and a set of related endogenous opioid peptides (EOPs), which show distinct selectivity toward their respective opioid receptors (ORs). Despite being key to the development of safer analgesics, the mechanisms of molecular recognition and selectivity of EOPs to ORs remain unclear. Scientists have systematically characterized the binding of EOPs to ORs and present five structures of EOP-OR-Gᵢ complexes, including β-endorphin- and endomorphin-bound μOR, deltorphin-bound δOR, dynorphin-bound κOR, and nociceptin-bound NOPR.

Psychoactive drugs and anesthetics work on brain. Narcotics work on brain and spinal cord. Nerve blocking anesthetics work on nerves, surface anesthetics and non-opioid anesthetics work on peripheral pain receptors. There are three types of opiate receptors and their simulation effect as followed—1. Mu or μ—these are associated with supraspinal analgesia, respiratory depression, euphoria and drug dependency, 2. Kappa or κ—These are associated with spinal analgesia, miosis and sedation, and 3. delta or δ—these cause dysphoria, hallucination and stimulation of vasomotor center. Opiates have two kinds of activities—central and peripheral. The effects of central activity are—analgesia, sedation, tranquility, euphoria/dysphoria, respiratory depression, emetic/anti-emetic, miosis, anti-diuretic. The effects of peripheral activity are delayed gastric emptying due to pyloric constriction, reduced gastro-intestinal motility, contracted bile duct sphincter, increased bladder muscle tone, decreased vascular tone and increased skin reaction such as itching. Drugs can be full agonist or partial agonists for opiate receptors. A compound can be agonist for one receptor and antagonist for other. Naloxone is a full antagonist for opiate receptors. Opiates can have the following effects—Depressant effect on respiration causing obstructive lung disease and emphysema, hypotension, spasmatic constipation, urinary retention-overfilling of bladder, pupillary constriction and miosis. Naloxone, a widely recognized opioid antagonist, has been successfully used as an antidote to reverse opioid overdoses and save lives. Naloxone administration can pose severe withdrawal symptoms. The dose has to be reduced and the dosing interval need to be shortened.

Opioid Pure agonists: e.g. codeine, morphine, fentanyl II. Mixed agonist/antagonist opioid drugs: e.g. butorphanol, nalbuphine, pentazocine, buprenorphine III.

Opioid antagonists: e.g. naloxone, naltrexone, methylnaltrexone. Opioid antagonists are medications that bind to opioid receptors in the brain, effectively blocking the effects of opioids, such as euphoria and analgesia.

Drug addiction starts with caffeine and nicotine (cigarettes and other tobacco products), alcohol, opioids (OxyContin, Heroin, Fentanyl, alfentanil, sufentanil, hydrocodone, codeine, morphine, oxymorphone, meperidine, pentazocine), stimulants (Cocaine, methamphetamine), hallucinogens (LSC and PCP), inhalants (paint fumes, glue), Benzodiazepines (Valium, Xanax), Barbiturates (phenobarbital, secobarbital), Marijuana and anabolic steroids etc.

Heroin and cocaine cause a rush of dopamine resulting in euphoria. Barbiturates and benzodiazepines slow down brain function resulting in calming of central nervous system. Methadone is a man-made opiate used to treat heroin addiction. However, it is highly addictive and should not taken for longer than 3 months to prevent addiction. Methadone can cause respiratory problems. Meth and crystal meth are CNS stimulants. Morphine, codeine etc. are pain-killers but also create a feeling of euphoria and make one get addicted. Anticonvulsant medication, gabapentin, is not as addictive as heroin, but it helps people to relax, reduce anxiety by affecting CNS. Cannabis is believed to be non-addictive.

Naloxone is an opiate antagonist used to reverse the respiratory depression caused by overdose of heroin, morphine etc. Naloxone reverses the effects of opioid analgesics by binding to the opioid receptors in the CNS and inhibit analgesia, euphoria, respiratory depression and bradycardia. Naloxone binds with the mu-opioid receptors with high affinity and a lesser degree to kappa- and gamma-opioid receptors in the CNS.

Naloxone is available as injection (0.4 mg/ml, 1 mg/mL), IM injection (5 mg/0.5 mL), nasal spray (8 mg/spray and 10 mg/spray), metered nasal spray (4 mg/spray), OTC nasal spray (Narcan, 4 mg/spray), Tablets of naloxone and pentazocine (0.5 mg base and 50 mg base), buccal/sublingual films of buprenorphine HCl/Naloxone HCl (2 mg/0.5 mg, 4 mg/l mg, 8 mg/2 mg, 12 mg/3 mg), sublingual tablets of buprenorphine/naloxone (2 mg/0.5 mg, 8 mg/2 mg), Zubsolv tablets 0.7 mg/0.18 mg, 1.4 mg/0.36 mg, 2.9 mg/0.71 mg, 5.7 mg/1.4 mg, 8.6 mg/2.1 mg, 11.4 mg/2.9 mg).

Pentazocine is the agonist of kappa opiate receptors and partial agonist of mu opiate receptors in the CNS. It produces analgesia, respiratory depression and sedation similar to other opioids. Buprenorphine is an opioid used to treat opioid use disorder, acute pain and chronic pain. It can cause respiratory depression, sleepiness, QT prolongation, low blood pressure, allergic reaction and opioid addiction.

In combination with naloxone, buprenorphine helps to treat people with opioid use disorder. Naloxone causes acute withdrawal symptoms. Before starting buprenorphine, individuals are generally advised to wait long enough after their last dose of opioid until they have some withdrawal symptoms to allow for the medication to bind the receptors, since if taken too soon, buprenorphine can displace other opioids bound to the receptors and precipitate an acute withdrawal. The dose of buprenorphine is then adjusted until symptoms Improve, and individuals remain on a maintenance dose of 8-16 mg. Combination of buprenorphine and naloxone can: treat opioid/heroin cravings, reduce severity of opioid withdrawal symptoms and treat opioid addiction as a medication-assisted treatment. Buprenorphine binds to the same opioid receptors as full opioid agonist but activates them much more slowly and not in full producing a weaker effect. It produces lesser euphoric effect.

Flumazenil injection (0.5 mg/0.5 mL, 1 mg/10 mL) has been approved in the US. Flumazenil is a selective GABA receptor antagonist administered via injection, intranasally or by Otic insertion. It acts as an antagonist and antidote to benzodiazepines through competitive inhibition. It has a short half-life (effect is observed in 2-3 minutes) and need multiple doses. There is potential risk of severe withdrawal symptoms.

Fentanyl is an approved analgesic. It is a highly potent synthetic opioid 50 to 100 time more potent than morphine and it is used in pain management and sedation. It is one of the commonly abused drugs, used by smoking, sniffing, patches, injection or tablets. US government has taken up a war against fentanyl as it kills more people than car accidents, cancer etc. The purity of illegal fentanyl is questionable and it is getting adulterated with other dangerous substances such as xylazine.

A. Pettersson (U.S. Pat. Nos. 8,470,361 and 8,658,198) invented a particulate transmucosal pharmaceutical composition in the form of a tablet for sublingual or buccal administration comprising a pharmacologically-effective amount of an opioid analgesic (fentanyl) and an opioid antagonist (naloxone).

Overall, there are products commercially available containing opioid agonist and opioid antagonist. Opioid agonist manages the withdrawal symptoms during opioid detoxification. Abrupt reversal of opioid depression using naloxone may result in nausea, vomiting, sweating, tremulousness, tachycardia, hypotension, hypertension, seizures, ventricular tachycardia and fibrillation, pulmonary edema, cardiac arrest, death, coma, and encephalopathy

Xylazine is reported to be adulterant in an increasing number of illicit drugs and has been found in a growing number of overdose deaths. Xylazine is a drug used for sedation, anesthesia, and muscle relaxation in animals such as horses and cattle. It has not been approved for human use. US lawmakers are moving to classify xylazine as a controlled substance. It is an analog of clonidine and an agonist of alpha-2 adrenergic receptors. Fentanyl is fast acting opioid. People who use it claim that the addition of xylazine to fentanyl extends the duration of staying high. Xylazine is associated with severe soft-tissue wounds and necrosis and can lead to amputation. Xylazine is not an opioid and therefore, naloxone does not address the impact of xylazine on breathing. Thus, naloxone does not act as an antidote for xylazine or xylazine tainted fentanyl.

Tramadol is another approved opioid used in pain management in patients for whom other treatments do not work. Tramadol has been declared as a controlled drug substance by DEA in 2014. It has serious and deadly side effects.

Tiletamine is used for anesthesia and it is classified as NMDA receptor antagonist. It is used in veterinary medicine in combination with zolazepam. Zolazepam is a pyrazolodiazepinone derivative structurally related to the benzodiazepine drugs, which is used as an anesthetic for a wide range of animals in veterinary medicine. Zolazepam is usually administered in combination with other drugs such as the NMDA antagonist tiletamine or the alpha-2 adrenergic receptor agonist xylazine, depending on what purpose it is being used for.

Atipamezole is an alpha-2 antagonist that was developed to reverse the actions of medetomidine and dexmedetomidine (non-narcotic alpha-2 agonists) in animals. One needs the doses of atipamezole to be five times and ten times that of medetomidine and dexmedetomidine, respectively. Humans are less sensitive to the effects of atipamezole when used as an antagonist for dexmedetomidine. Atipamezole reverses cardiopulmonary effects of alpha-2 agonists in dogs. Potential adverse effect of atipamezole in dogs are diarrhea, hypersalivation, and tremors. Yohimbine and tolazoline are also used to reverse the effects of xylazine. Yohimbine and tolazoline are non-specific alpha-2 antagonist compared to atipamezole. They can produce hypotension and reflex tachycardia. Tolazoline may produce histamine release, contributing to hypotension. Novartis obtained an approval for Tolazoline injection, 25 mg/ml (Priscoline) on Feb. 22, 1985. But it has been now discontinued. Intramuscular administration of atipamezole produces rapid and complete reversal of alpha-2 agonist effects. The IV bolus administration might be reserved for emergency or life-threatening situations.

U.S. Pat. No. 8,475,832 (2013) relates to a film dosage form comprising: a polymer carrier matrix, a therapeutic effect amounts of buprenorphine and naloxone, and a buffer in an amount to provide a local pH for said composition of a value sufficient to optimize absorption of buprenorphine. U.S. Pat. No. 11,135,216 is from the same inventors and containing same two drugs.

Autonomic nervous system is divided into-sympathetic and parasympathetic. Stimulation of sympathetic system increases heart rate, relaxation of bronchial muscles, contraction of gastrointestinal and urinary bladder sphincters, contraction of pupil, dilation of coronaries, skeletal muscle vessels etc. There are two kinds of adrenergic receptors-alpha (alpha 1 and 2), and Beta (beta 1, 2 and 3). Epinephrine and norepinephrine work on mainly alpha receptors. Isoproterenol works mainly on beta receptors. Norepinephrine also interacts with beta-1 receptors.

Antisedan formulation by Orion Corporation contains 5.0 mg/ml of atipamezole Hydrochloride. It is for intramuscular use in dogs only as a reversing agent of dexmedetomidine and medetomidine. Each mL contains 5.0 mg atipamezole HCl, 1.0 mg methyl paraben, 8.5 mg sodium chloride and water for injection. It is administered intramuscularly for reversal of sedation and analgesia. The atipamezole dose for the reversal of IV DEXDOMITOR (dexmedetomidine HCl) or DOMITOR (medetomidine) is 3750 mcg/m². The atipamezole dose for the reversal of IM DEXDOMITOR or DOMITOR is 5000 mcg/m². The dose is calculated based on body surface area/body weight.

Naloxone is widely used as an antidote for opioid over-dose; however, it is not effective against all the illicit drug such as fentanyl tainted with xylazine. Yohimbin is a presynaptic alpha-2 adrenergic receptor blocker. Xylazine is alpha adrenergic receptor agonist. Naloxone does not work on overdoses caused by drugs that aren't opioids.

The combination of opioid antagonists and alpha-2 receptor antagonists seeks to capitalize on the unique properties of each class of medications to address different aspects of drug addiction. By blocking the opioid receptors, opioid antagonists like naloxone and naltrexone can help prevent the rewarding effects of opioids and reduce cravings. However, these medications alone may not effectively address other symptoms associated with addiction, such as anxiety, agitation, and sympathetic overactivity. By combining these two classes of medications, the aim is to provide a comprehensive approach to drug addiction reversal. The opioid antagonist component helps reduce opioid cravings and blocks the reinforcing effects of opioids, while the alpha-2 receptor antagonist component addresses withdrawal symptoms and sedation, potentially promoting abstinence and facilitating the recovery process. One has to consider factors such as the severity of addiction, patient characteristics, and the specific substances involved will be crucial for maximizing effectiveness. One should be integrated with comprehensive addiction treatment approaches. Combining medications with behavioral therapy, counseling, and social support is often the most effective strategy for long-term recovery and relapse prevention.

Clonidine is an alpha-2 receptor antagonist that has been used off-label to manage withdrawal symptoms associated with opioid or alcohol dependence. It helps alleviate symptoms such as anxiety, agitation, and high blood pressure. Clonidine is available in oral formulations, typically used in combination with other medications in addiction treatment.

It's important to remember that the combination of opioid antagonists and alpha-2 receptor antagonists for drug addiction reversal is still an area of ongoing research and not yet a standard treatment approach. There is currently a need for formulations containing an opiate antagonist and alpha-2 antagonist providing the desired levels of both compounds. Naloxone is a μ-opioid antagonist, which blocks the effects of opioids. Atipamezole is an alpha-2 adrenoreceptor antagonist. It is rapidly absorbed and distributed from periphery to central nervous system. By combining naloxone and atipamezole, we can combat respiratory depression and sedation caused by opioids. It is necessary to titrate the doses of both on case-by-case basis and situation, and for that, there is a need to develop different dosage forms with multiple strengths.

Based on the discussion above and current opioid crisis in the US and in many other countries, there is an urgent need of an antidote such as Naloxone plus Atipamezole combination. This will save lives of hundreds of drug addicts or accidental drug addicts. We have invented several dosage forms of combination of naloxone and atipamezole so that a treatment will be available in any situation.

### SUMMARY OF THE INVENTION

This study provides several combination dosage forms comprising of naloxone or pharmaceutically acceptable salt thereof and atipamezole or pharmaceutically acceptable salt thereof to treat opioid dependence of an addict. In this document, a reference to naloxone or atipamezole also includes suitable salts thereof.

The combination therapy contains therapeutically effective amounts of Naloxone or its salts and Atipamezole or its salts can be variable based on the need of patients and their conditions. The dose of both actives varies based on the dosage form used. The concentration of naloxone and its salts in a dosage form is from 0.00001% to 5%. The concentration of atipamezole or its salts in a dosage form is from 0.00001% to 25%. Several dosage forms of the combination of naloxone and atipamezole were developed to be used via different routes of administration. Based on the bioavailability of both drugs for different routes of administration, the strengths of both drugs will be adjusted.

In an embodiment, an aqueous solution of naloxone and atipamezole has a pH in the range 3 to 4 and the injection formulation is injected intravenously.

In another embodiment, the present invention, directed to immediate release injection formulation also comprising about 0.0001% to 1.0% w/w of a chelating agent, preferably edetate disodium dihydrate.

In another embodiment, a sustained release injection formulation of naloxone and atipamezole combination is administered intramuscularly or subcutaneously. The formulations may have a depot effect. The depot formulations are expected to release the drugs to blood from the injection site over days, weeks and months. The weight ratio of naloxone to atipamezole is from 5:0.00001 to 0.00001:25%.

In an embodiment, the topical gel formulations of naloxone and atipamezole combination therapy is administered on the skin or in a naso-pulmonary region.

In an embodiment, the immediate release tablet contains a combination of naloxone and atipamezole. These tablets are administered orally.

In another embodiment, the tablet containing naloxone and atipamezole is administered sublingually.

In another embodiment, the tablet containing naloxone and atipamezole is administered sublingually and it is an orally disintegrating tablet.

In another embodiment, the tablet of naloxone and atipamezole is an effervescent tablet.

In another embodiment, the tablet of naloxone and atipamezole is a gelling tablet. The tablet is placed in a spoon. Sufficient amount of water is added on the tablets. The tablet absorbs water, swells and forms a gel. The gel is smooth and can be swallowed easily without an additional quantity of water.

In another embodiment, a film dosage form comprises of a combination of naloxone and atipamezole and the film is administered orally. The film dissolves in the mouth quickly.

A method of treating drug addict consisting the steps of: a) providing a combination dosage form composition consisting: i) a therapeutically effective amount of naloxone or a pharmaceutically acceptable salt thereof; ii) a therapeutically effective amount of atipamezole or a pharmaceutically acceptable salt thereof; and b) administering said composition parenterally.

A parenteral dosage form of naloxone and atipamezole further comprises a chelating agent.

A parenteral dosage form of naloxone and atipamezole is administered intravenously.

A parenteral dosage form of naloxone and atipamezole is administered intramuscularly.

A parenteral dosage form of naloxone and atipamezole is administered subcutaneously.

A method of treating drug addict consisting the steps of: a) providing a combination drug dosage form composition consisting: i) a therapeutically effective amount of naloxone or a pharmaceutically acceptable salt thereof; ii) a therapeutically effective amount of atipamezole or a pharmaceutically acceptable salt thereof; and b) administering said composition orally.

An oral dosage form of naloxone and atipamezole is an immediate release tablet or a capsule or a film.

An oral dosage form of naloxone and atipamezole is a sublingual tablet.

An oral dosage form of naloxone and atipamezole is an orally disintegrating tablet.

An oral dosage form of naloxone and atipamezole is a sublingual orally disintegrating tablet.

An oral dosage form of naloxone and atipamezole is an effervescent tablet.

An oral dosage form of naloxone and atipamezole is a gelling tablet.

An oral dosage form of naloxone and atipamezole is an orally dissolving film.

A method of treating a drug addict consisting the steps of: a) providing a combination dosage form composition consisting: i) a therapeutically effective amount of naloxone or a pharmaceutically acceptable salt thereof; ii) a therapeutically effective amount of atipamezole or a pharmaceutically acceptable salt thereof; and b) administering said composition topically.

A topical dosage form of naloxone and atipamezole further comprises a chelating agent.

A topical dosage form of naloxone and atipamezole is administered in the naso-pulmonary region.

A topical dosage form of naloxone and atipamezole is administered topically on the skin.

A topical dosage form of naloxone and atipamezole is a clear solution.

A topical dosage form of naloxone and atipamezole is a gel.

### DETAILED DESCRIPTION

### Definition of Terms Used

A drug or medicine is “a chemical substance used in the treatment, cure, prevention, or diagnosis of disease or used to otherwise enhance physical or mental well-being. Drugs may be prescribed for a limited duration, or on a regular basis for chronic disorders. Medicament is a medicine, or a substance used in a therapy.

There is another meaning to the word “drugs” on the street. They are also termed as “street drugs”. These are psychoactive substances, especially which are illegal and addictive. These are ingested for recreation use to get “high”. Any drug that can be abused and cause addiction ends up as a street drug. The most common street drugs are substances such as marijuana, delta 8 THC, opioids, over the counter medications, prescription medicines such as oxycodone, alcohol, steroids, cocaine, fentanyl, hallucinogens, heroin, and many more. According to the CDC (centers for disease control), 107,735 Americans died between August 2021 and August 2022 from drug poisonings, with 66 percent of those deaths involving synthetic opioids like fentanyl.

A “medicinal agent” is a pharmaceutical agent that includes drugs, biological products, and medical devices under the regulatory authority of the Food and Drug Administration (FDA). A “medicinal agent” shows pharmacological activity in human subjects and can be “allopathic drug molecules” or “natural drug products”.

“Dosage Form” is the physical form of a dose of a chemical compound used as a drug or medication intended for administration or consumption. A “compound or an active pharmaceutical ingredient (API)” is a drug substance with intended medicinal action.

“Formulation” is the process in which different chemical substances, including the active drug(s), are combined to produce a final medicinal product or a dosage form. In a combination formulation, two or more actives or drugs are included in the dosage form.

A parenteral administration or route is defined as administration of drugs as a liquid into the blood by bypassing the gastrointestinal system. The dosage form can be administered in the blood (intravenously), in the muscle (intramuscularly), under the skin (subcutaneously) or other part of the body.

An oral administration is a route of administration of the medicines which is taken through the mouth, swallowed so that it reaches the gastrointestinal system, processed so that the drug undergoes systemic absorption. A systemic absorption refers to absorption into a circulatory system. A circulatory system be blood or lymphatic fluid.

Topical formulation”—These formulations are applied directly to a part of the body. Generally, topical formulations are perceived to be applied on skin where the drug action is needed. Many nasal delivery systems are considered topical formulations too when the effect is desired in the naso-pulmonary area.

Thus, the dosage forms can be administered parenterally, orally or topically.

Excipient refers to additives useful for converting pharmacologically active compounds into pharmaceutical dosage forms, which are suitable for administration to patients. Suitable excipients include diluents, chelating agent, binders, disintegrants, surfactants, lubricants, glidants and coloring agents.

“Diluent”—Diluent selected are water-soluble and water-insoluble. Suitable water-soluble diluents include sucrose, dextrose, lactose, mannitol, sorbitol and the like and water-insoluble diluents include starch, microcrystalline cellulose, silicified microcrystalline cellulose, calcium silicate and the like or combination thereof.

“Chelating agent”—The term “chelating agent,” as used herein, means a molecule containing two or more electron donor atoms that can form coordinate bonds to a single metal ion. The term “chelating agent” is understood to include the chelating agent as well as salts thereof. For example, the term “chelating agent” includes citric acid as well as its salt forms. The most common and widely used chelating agents coordinate to metal atoms through oxygen or nitrogen donor atoms, or both. Other less common chelating agents coordinate through sulfur in the form of —SH (thiol or mercapto) groups.

“Disintegrating agent” Disintegrants are used to break apart a tablet into small granules. Suitable disintegrants used according to the present invention are selected from starch, cross-povidone, sodium starch glycolate, croscarmellose sodium and the like or combination thereof.

“Binder” are substances, which bind different particles to form a mass. Suitable binders used according to the present invention are selected from the group comprising of hydroxypropyl methylcellulose, maize starch, povidone, hydroxypropyl cellulose, pregelatinized starch and the like or combination thereof.

“Lubricant or glidant” lubricants are the agents added to tablet and capsule formulations to improve the powder processing properties of formulations. Suitable lubricants used according to the present invention are metallic salts of fatty acids such as magnesium stearate and stearic acid. Other lubricants can be used are Fatty acid esters, including glyceride esters (glyceryl monostearate, glyceryl tribehenate, and glyceryl dibehenate) and sugar esters (sorbitan monostearate and sucrose monopalmitate) and inorganic material as talc or mixtures thereof.

To mask the taste, sweeteners are commonly used. Sweeteners that can be used include acesulfame potassium, aspartame, aspartame-acesulfame salts, cyclamate, erythritol, glycerol, glycyrrhizin, hydrogenated Starch hydrolysate, isomalt, lactitol, maltitol, mannitol, neotame, polydextrose, Saccharin, Sorbitol. Sucralose, tagatose, Xylitol, dextrose, glucose, fructose, and honey or mixtures thereof.

“Film forming polymers” are used to manufacture “film” formulations. Suitable film coating polymers used according to the present invention are selected from povidone, ethyl cellulose, hydroxypropyl cellulose, hydroxypropyl methylcellulose, polyvinyl alcohol and the like or mixtures thereof.

“Gelling polymers” are those which form a gel when a solvent is added to the polymer. Gelling agents selected from the group consisting of xanthan gum, locust bean gum, gellan gum, guar gum, pullulan, konjac flour, kappa-carrageenan, iota-carrageenan, gum tragacanth, and combinations thereof; and water.

“Immediate release” dosage form—As the name suggest, the release of drugs from the dosage form takes place immediately, within few minutes. Injectable solutions mix with the blood right away thereby making the drug available. In the case of tablets, the tablet has to first disintegrate to produce granules. The drugs are then released in the fluids in the Gastro-intestinal tract so that they can be absorbed. The definition does not describe an immediate absorption of the drug. It defines an immediate release of the drug from the dosage form.

“Sustained release” refers to the release of an active ingredient over an extended period of time. There are two aspects—1. Slow rate of release of drugs from the dosage form, 2. Release of entire quantity of drug loaded in the dosage form.

“Nasal delivery” refers to formulations that are delivered to the naso-pulmonary area. It is delivered in the nose, throat, trachea and lungs.

“Oral delivery” refers to the route of absorption through the digestive tract for local or systemic therapy. Drug formulations are orally delivered to reach the buccal cavity, oesophagus, stomach and intestine.

“Sublingual tablets”—As the name suggests, the tablets are placed under the tongue. The drug is released from the tablets and a portion of drug is absorbed from the mouth cavity. The remaining portion of the dosage form passes on to the GI tract.

“Orally disintegrating tablets (ODT)”—As the name suggests, the tablet disintegrates in the mouth cavity within few seconds. The drug may or may not be absorbed from the mouth cavity. The patient does not need water or drink to swallow the tablet.

“Sublingual ODT”—This is a combination of the above two concept. The tablet is kept under the tongue and it will disintegrate in few seconds (less than 30 seconds).

“Effervescent tablets”—Effervescent means giving off bubbles or fuzz. The effervescent has a lively quality or a perspective of filled with energy. The fuzz also helps to disintegrate the tablet. It may not help drug absorption but has an knack to appeal patients.

“Gelling tablets”—Gelling is a semi-rigid jelly-like colloid in which a liquid is dispersed in a solid. A tablet, when added to a small amount of water, in a spoon or a small container, absorbs water, swells and forms a soft get, which a patient can swallow easily without additional water.

A combination dosage form contains more than one drug substance. In this case, we are combining naloxone an atipamezole.

Drug addiction or substance use disorder is a disease that affects a person's brain and behavior and leads to an inability to control the use of a legal or illegal drug or medicine. Drugs or medicines in this context are heroin, cocaine, ecstasy, flakka, LSD, marijuana, methamphetamines, mushrooms, fentanyl etc. It can include opioid painkillers, alcohol, nicotine etc. too. A person who is addicted to such drugs is termed as a “drug addict”. Recently, fentanyl adulterated with xylazine has created a huge problem to drug addicts.

Following examples are illustrative of present invention. The concentration of naloxone in the formulations can be from 0.01 mg/ml to 25 mg/mL. The concentration of atipamezole is from 0.01 mg/mL to 100 mg/mL.

### Immediate Release Injectable Formulation:

### Aqueous Injectable Solution

Aqueous solutions with 1 mg/m concentrations of naloxone and atipamezole in prefilled syringes were examined. Batch 1 contained only Naloxone, 1 mg/ml. Batch 2 contained only Atipamezole, 1 mg/mL. Batch 3 contained both naloxone and atipamezole at 1 mg/mL concentrations. Batches 5, 6 and 7 did not contain sodium EDTA (ethylene diamine tetraacetic acid).

TABLE 1
Naloxone and Atipamezole formulations with EDTA

| | | | | | Naloxone + | | | |
|---|---|---|---|---|---|---|---|---|
| | Naloxone | | Atipamezole | | Atipamezole | | Placebo | |
| Component | Batch 1 | | Batch 2 | | Batch 3 | | Batch 4 | |
| Naloxone, HCl | 1 | mg/mL | — | | 1 | mg/mL | — | |
| Atipamezole | — | | 1 | mg/mL | 1 | mg/mL | — | |
| Sodium Chloride | 800 | mg | 800 | mg | 800 | mg | 800 | mg |
| EDTA Sodium | 20 | mg | 20 | mg | 20 | mg | 20 | mg |

| | |
|---|---|
| 1N HCl | pH 3-3.4 |

TABLE 2
Naloxone and Atipamezole formulations w/o EDTA

| | | | | | Naloxone + | |
|---|---|---|---|---|---|---|
| | Naloxone | | Atipamezole | | Atipamezole | |
| Component | Batch 5 | | Batch 6 | | Batch 7 | |
| Naloxone, HCl | 1 | mg/mL | — | | 1 | mg/mL |
| Atipamezole | — | | 1 | mg/mL | 1 | mg/mL |
| Sodium Chloride | 800 | mg | 800 | mg | 800 | mg |

| | |
|---|---|
| 1N HCl | pH 3-3.4 |

Both drugs (Naloxone and Atipamezole) were weighed and added to the volumetric flasks. Water, 70% of total volume, was added into the volumetric flasks to dissolve the drugs. Drugs dissolved in a short time (˜2-3 mins). Weighed and added the remaining ingredients to the flasks. More (˜20%) water was added to the flasks. The samples were ultrasonicated until all ingredients dissolved completely. The initial pH was measured. It was about 4.7 for all the formulations. Adjusted the pH to 3-3.5 using 1 N HCl, and made the up volume. Measured the final pH for all the formulations. Found the densities for the formulations and details are listed in the table below (Table 3).

TABLE 3
Densities of various Naloxone - Atipamezole
formulations and their pH's.

| | Density | |
|---|---|---|
| Formulation # | (gm/mL) | pH |
| #1- Naloxone HCl (1 mg/mL) - EDTA | 1.0014 | 3.14 |
| #2- Atipamezole (1 mg/mL) - EDTA | 1.0006 | 3.28 |
| #3- Naloxone + Atipamezole (1 mg/mL) - EDTA | 1.0021 | 3.14 |
| #4- Placebo - EDTA | 1.0016 | 3.28 |
| #5- Naloxone (1 mg/mL)- no EDTA | 1.0021 | 3.17 |
| #6- Atipamezole (1 mg/mL)- no EDTA | 1.0015 | 3.26 |
| #7- Naloxone, Atipamezole (1 mg/mL) | 1.0013 | 3.21 |

All the formulations were divided and stored at different stability conditions (25° C., 40° C. and 60° C.). Tables 4, 5, 6, 7, 8 and 9 lists the % assay values of various batches at different stability conditions. Overall, both compounds in independent formulations and as combination products were stable. In general, based on the assay and percent peak area values, we concluded that EDTA in the formulations made them more stable and will be included in the future formulations.

TABLE 4
Naloxone HCl- Atipamezole Stability data for the Formulations with EDTA at 25° C.

| | | 25° C., % assay values | | | | | |
|---|---|---|---|---|---|---|---|
| | Zero | Week | Week | Week | Week | 2 | 4 |
| Percent Assay | time | 1 | 2 | 3 | 4 | Month | Month |
| B #1 Naloxone_1 mg/mL | 100.2 | 98.4 | 99.0 | 98.7 | 99.4 | 101.6 | 99.5 |
| B #2 Atipamezole_1 mg/mL | 99.3 | 99.6 | 101.4 | 99.5 | 100.7 | 100.1 | 105.2 |
| B #3 NaI + Atp_1 mg/mL_Naloxone | 100.5 | 98.5 | 99.6 | 100.2 | 98.7 | 98.8 | 97.5 |
| B #3 NaI + Atp_1 mg/mL_Atipamezole | 99.4 | 98.2 | 99.4 | 100.6 | 100.7 | 99.7 | 106.1 |

TABLE 5
Naloxone HCl- Atipamezole Stability data for the Formulations with EDTA at 40° C.

| | | 40° C., % assay values | | | | | |
|---|---|---|---|---|---|---|---|
| | Zero | Week | Week | Week | Week | 2 | 4 |
| Percent Assay | time | 1 | 2 | 3 | 4 | Month | Month |
| B #1 Naloxone_1 mg/mL | 100.2 | 99.4 | 99.4 | 101.7 | 99.9 | 99.0 | 98.9 |
| B #2 Atipamezole_1 mg/mL | 99.3 | 100.5 | 100.2 | 98.8 | 98.3 | 101.3 | 103.1 |
| B #3 NaI + Atp_1 mg/mL_Naloxone | 100.5 | 99.5 | 101.1 | 99.9 | 98.1 | 98.5 | 100.7 |
| B #3 NaI + Atp_1 mg/mL_Atipamezole | 99.4 | 99.6 | 100.4 | 99.9 | 99.5 | 99.7 | 103.1 |

TABLE 6
Naloxone HCl- Atipamezole Stability data for the Formulations with EDTA at 60° C.

| | | 60° C., % assay values | | | | | |
|---|---|---|---|---|---|---|---|
| | Zero | Week | Week | Week | Week | 2 | 4 |
| Percent Assay | time | 1 | 2 | 3 | 4 | Month | Month |
| B #1 Naloxone_1 mg/mL | 100.2 | 100.6 | 101.2 | 101.2 | 100.3 | 99.0 | 99.0 |
| B #2 Atipamezole_1 mg/mL | 99.3 | 102.2 | 100.9 | 101.3 | 100.2 | 101.3 | 97.2 |
| B #3 NaI + Atp_1 mg/mL_Naloxone | 100.5 | 101.7 | 100.4 | 99.4 | 98.5 | 97.5 | 98.6 |
| B #3 NaI + Atp_1 mg/mL_Atipamezole | 99.4 | 101.5 | 100.2 | 99.8 | 99.7 | 100.5 | 102.3 |

TABLE 7
Naloxone HCl-Atipamezole Stability data for the formulation's w/o EDTA at 25° C.

| | | 25° C., % assay values | | | | |
|---|---|---|---|---|---|---|
| | Zero | Week | Week | Week | Week | 3 |
| Percent Assay | time | 1 | 2 | 3 | 4 | Month |
| B #5 Naloxone_1 mg/mL_w/o EDTA | 100.7 | 100.8 | 100.5 | 102.4 | 102.1 | 98.2 |
| B #6 Atipamezole_1 mg/mL_w/o EDTA | 100.5 | 99.3 | 103.1 | 99.9 | 100.4 | 103.2 |
| B #7 NaI + Atp_1 mg/mL_Naloxone_w/o EDTA | 101.7 | 99.9 | 100.8 | 99.9 | 98.9 | 96.9 |
| B #7 NaI + Atp_1 mg/mL_Atipamezole_w/o EDTA | 99.5 | 97.6 | 110.9 | 101.9 | 95.4 | 100.6 |

TABLE 8
Naloxone HCl-Atipamezole Stability data for the formulation's w/o EDTA at 40° C.

| | | 40° C., % assay values | | | | |
|---|---|---|---|---|---|---|
| | Zero | Week | Week | Week | Week | 3 |
| Percent Assay | time | 1 | 2 | 3 | 4 | Month |
| B #5 Naloxone_1 mg/mL_w/o EDTA | 100.7 | 100.5 | 101.4 | 99.6 | 98.9 | 95.7 |
| B #6 Atipamezole_1 mg/mL_w/o EDTA | 100.5 | 99.6 | 102.4 | 102.7 | 99.4 | 100.9 |
| B #7 NaI + Atp_1 mg/mL_Naloxone_w/o EDTA | 101.7 | 99.7 | 101.3 | 100.1 | 99.7 | 96.7 |
| B #7 NaI + Atp_1 mg/mL_Atipamezole w/o EDTA | 99.5 | 96.9 | 101.7 | 101.2 | 96.5 | 99.4 |

TABLE 9
Naloxone HCl-Atipamezole Stability data for the formulation's w/o EDTA at 60° C.

| | | 60° C. | | | | |
|---|---|---|---|---|---|---|
| | Zero | Week | Week | Week | Week | 3 |
| Percent Assay | time | 1 | 2 | 3 | 4 | Month |
| B #5 Naloxone_1 mg/mL_w/o EDTA | 100.7 | 99.2 | 99.5 | 98.6 | 96.3 | 91.7 |
| B #6 Atipamezole_1 mg/mL_w/o EDTA | 100.5 | 101.4 | 102.4 | 101.9 | 98.1 | 102.3 |
| B #7 NaI + Atp_1 mg/mL_Naloxone_w/o EDTA | 101.7 | 101.6 | 98.8 | 97.9 | 96.9 | 91.9 |
| B #7 NaI + Atp_1 mg/mL_Atipamezole w/o EDTA | 99.5 | 99.5 | 101.3 | 101.0 | 97.3 | 98.2 |

### Sustained Release IM (Intramuscular) and Subcutaneous Injectable Formulations

We prepared two sustained release formulations of naloxone and atipamezole using either PEG 400 (polyethylene glycol 400) or xanthan gum or sodium CMC (carboxymethyl cellulose). Table 10 lists the composition containing PEG 400.

Sustained Release Formulation with PEG 400

TABLE 10
Sustained release formulation of Naloxone: Atipamezole

| Ingredient | Percent |
|---|---|
| Naloxone HCl | 0.5 |
| Atipamezole | 0.5 |
| Sodium Chloride | 0.75 |
| PEG 400 | 10 |
| Di sodium EDTA | 0.020 |
| Water | q.s. to 100% |
| 0.1N HCl | q.s. to pH 3.5 |

Naloxone drug substance was weighed into an appropriate container and water was added to dissolve the drug. Atipamezole was added to the same container along with some more water and mixed. Both drugs dissolved immediately. Added sodium chloride, disodium EDTA and PEG 400. Mixed and checked pH. We used 0.1 N HCl to adjust pH to ˜3.5.

Both the injectable formulations (immediate and sustained release) can be administered using a normal syringe or can be administered via an autoinjector. A normal syringe is the one used by medical professional to administer medicines to patients. An autoinjector is a medical device designed to deliver a dose of particular drug to either intramuscular or subcutaneous site.

Sustained release formulation with sodium carboxymethyl cellulose (Na CMC) Table 11 lists the composition of sustained release formulation of naloxone and atipamezole.

TABLE 11
Sustained release formulation of Naloxone: Atipamezole

| Ingredient | Percent |
|---|---|
| Naloxone HCl | 0.5 |
| Atipamezole | 0.5 |
| Sodium Chloride | 0.75 |
| Sodium CMC | 0.05 |
| EDTA | 0.020 |
| H2O | q.s. to 100% |
| 0.1N HCl | q.s. to pH 3.5 |

Weighed Sodium CMC to the container and added some water to dissolve Mixed, Sodium CMC dissolved easily. Added Naloxone and mixed. Added atipamezole along with some more water and mixed. Drugs dissolved. Added NaCl and EDTA. Mixed and checked the pH. Used 0.1 N HCl to adjust pH to 3.5.

### Topical Application for Nasal and Skin Delivery

People, high on drugs, may not be able to take an oral delivery of naloxone-atipamezole combination. One may not have an injectable dosage form available. Thus, it was felt necessary to develop a topical application of the combination of naloxone-atipamezole. Composition in Table 12 contained a gel product in an aqueous phase. The gel was formed using sodium hyaluronate.

Nasogel—with More Aqueous Phase

TABLE 12
Nasolgel formulation of Naloxone:
Atipamezole with hyaluronic acid

| Ingredient | Percent |
|---|---|
| Naloxone HCl | 0.05 |
| Atipamezole | 0.5 |
| Sodium Chloride | 0.75 |
| Disodium EDTA | 0.02 |
| Hyaluronic Acid as a sodium salt | 1 |
| H2O | q.s. to 100% |
| 0.1N NaOH/0.1N HCl | q.s. to pH 5 |

Sodium hyaluronate was weighed into the container and some water added to dissolve. Hyaluronic acid dissolved easily and formed a nice gel. In a separate container, Naloxone and atipamezole were added to water and mixed. Both the drugs dissolved easily. Sodium chloride and sodium EDTA were added and mixed. More water was added to make up the volume. Added the aqueous solution to the hyaluronic acid gel and mixed. Rinsed the aqueous solution container with water and added the rinsate to the product. Mixed the sample and the pH was measured. Adjusted the pH to 5.

By reducing the amount of hyaluronic acid in the formulation to 0.1%, a clear solution formulation of Naloxone:Atipamezole was prepared.

Topical Gel—with Ethanol

Table 13 lists the composition of product containing less water than formula in Table 12. It was replaced by 200 proof ethanol. Ethanol has been shown to increase the permeation of drugs across the skin barrier.

TABLE 13
Topical gel formulation with more ethanol

| Ingredient | Percent |
|---|---|
| Naloxone HCl | 0.5 |
| Atipamezole | 2.5 |
| Ethanol, 200 proof | 40 |
| Disodium EDTA | 0.02 |
| Transcutol | 10 |
| Xanthan gum | 0.2 |
| H2O | q.s. to 100% |
| 0.1N NaOH/0.1N HCl | q.s. to pH 5 |

Transcutol (diethylene glycol monoethyl ether) and ethanol were weighed into a suitable container and xanthan gum was added to dissolve. Once dispersed, water was added. The mixture was heated to dissolve xanthan gum. Once dissolved, Naloxone and atipamezole were added and the product was mixed well. The pH was adjusted to 5 using 0.1 N sodium hydroxide. Both drugs dissolved fully. Transcutol increases the permeation of drugs through the skin.

### Oral Drug Delivery Systems (DDS)

Oral DDS have been used most widely. There are various kinds of dosage forms administered by an oral route. We prepared different kinds of formulations with these two drug substances.

### Immediate Release Tablets

Table 14 lists the composition of immediate release tablets containing naloxone and atipamezole.

TABLE 14
Immediate release tablets

| Ingredient | Percent | |
|---|---|---|
| Naloxone HCl | 10 | mg/tablet |
| Atipamezole | 100 | mg/tablet |
| Microcrystalline cellulose | 55 | mg |
| Povidone K30 to prepare the binding | 10 | mg |
| solution | | |
| Disodium EDTA | 0.02 | |
| Mannitol | 41 | mg |
| Magnesium stearate | 2 | mg |

The tablet weight is about 100 to 110 mg. Povidone K30 in water was used as a binding solution.

### Sublingual ODT

Table 15 lists the composition of sublingual orally disintegrating tablet.

TABLE 15
Sublingual orally disintegrating tablet formulation

| Ingredient | Percent |
|---|---|
| Naloxone HCl | 1 mg per tablet |
| Atipamezole | 1 mg per tablet |
| Sodium Starch Glycolate | 15.0 |
| Microcrystalline cellulose | 4.6 |
| Disodium EDTA | 0.02 |
| Acesulfame K | 0.2 |
| Magnesium Stearate | 0.2 |
| Mannitol | 70.8 |
| Polyvinyl acetate | 4.0 |
| Povidone | 0.2 |
| Kollidon CL-SF | 5.0 |

The tablets are prepared using a wet-granulation method. Water was used to make granules, which were dried at 60° C. The tablet when kept under the tongue, it absorbs saliva easily and disintegrates in 20 to 40 seconds. The mass produced is easy to swallow.

### Effervescent Tablets

Tablet 16 lists the composition of the effervescent tablet containing sodium bicarbonate and citric acid. The tablet can be added in a small amount of water in a glass. The effervescence produced disintegrates the tablet. The suspended material is then swallowed. It is important to manufacture and store all effervescent tablets in the low humidity conditions.

TABLE 16
Effervescent tablet formulation with sodium bicarbonate

| Ingredient | Percent |
|---|---|
| Naloxone HCl | 1 mg per tablet |
| Atipamezole | 1 mg per tablet |
| Sodium Starch Glycolate | 13.5 |
| Microcrystalline cellulose | 4.6 |
| Disodium EDTA | 0.02 |
| Acesulfame K | 0.2 |
| Magnesium Stearate | 0.2 |
| Milled citric acid monohydrate | 7.5 |
| Sodium Bicarbonate | 9.0 |
| Mannitol | 58.7 |
| Kollidon CL-SF | 3.0 |
| Polyvinyl acetate | 3.0 |
| Povidone | 0.3 |

Effervescent tablets are prepared using calcium carbonate instead of sodium bicarbonate (Table 17).

TABLE 17
Effervescent tablet formulation with calcium carbonate

| Ingredient | Percent |
|---|---|
| Naloxone HCl | 1 mg per tablet |
| Atipamezole | 5 mg per tablet |
| Sodium Starch Glycolate | 12.0 |
| Microcrystalline cellulose | 4.6 |
| Disodium EDTA | 0.02 |
| Acesulfame K | 0.2 |
| Magnesium Stearate | 0.2 |
| Milled citric acid monohydrate | 7.5 |
| Calcium Carbonate | 10.5 |
| Mannitol | 58.7 |
| Kollidon CL-SF | 3.0 |
| Polyvinyl acetate | 3.0 |
| Povidone | 0.3 |

### Gelling Tablet

Table 18 lists the composition of such a gelling tablet. It is useful to people having difficulty in swallowing big tablets or people suffering from dysphagia.

TABLE 18
Gelling tablet formulation

| Ingredient | Percent |
|---|---|
| Naloxone HCl | 10 mg per tablet |
| Atipamezole | 10 mg per tablet |
| Sodium Starch Glycolate | 20 |
| Kollidon 90 F | 10 |
| Disodium EDTA | 0.02 |
| Microcrystalline cellulose | 19.6 |
| Acesulfame potassium | 0.2 |
| Sorbitol | 50.0 |
| Magnesium Stearate | 0.2 |

The tablet is placed in a tablespoon and water is added so that the tablet gets wet. Tablet absorbs water immediately; it swells and disintegrate in a short time. The tablet forms a thickened soft mass, which can be swallowed easily.

### Orally Disintegrating Films

Weight of each film containing naloxone and atipamezole is ˜100 mg. Table 19 lists the composition of gel to make the films. After drying, each film contains about 3-4% water.

TABLE 19
Composition of orally disintegrating films

| Ingredient | Percent |
|---|---|
| Naloxone HCl | 0.25 |
| Atipamezole | 0.5 |
| Hydroxypropyl methylcellulose E5 (HPMC E5) | 20.0 |
| Disodium EDTA | 0.001 |
| Glycerin | 1.2 |
| Aspartame potassium | 0.0225 |
| Peppermint oil | 0.05 |
| Water | q.s. to 100% |

We used 20% HPMC gel to prepare films. Thus, 84 mg HPMC E5 was dissolved in 0.42 ml of water in the formulation. Peppermint oil does not dissolve in the aqueous phase, but it gets dispersed as fine globules.

All above formulations contained different amounts of naxolone and atipamezole. These formulations are only representation of various approaches and the dose of each drug (therapeutic amounts) can be easily modified based on the clinical requirement. A person with ordinary skills in formulation development will be able to make these modified formulations easily.

### CONCLUDING REMARKS

Drug addiction is a major problem in current times. Fentanyl is one of the most potent compounds on the street. United States Drug Enforcement Administration (US DEA) reports a widespread threat of fentanyl mixed with xylazine. Following is the quote from the DEA website—“Xylazine is making the deadliest drug threat our country has ever faced, fentanyl, even deadlier,” said Administrator Milgram. “DEA has seized xylazine and fentanyl mixtures in 48 of 50 States. The DEA Laboratory System is reporting that in 2022 approximately 23% of fentanyl powder and 7% of fentanyl pills seized by the DEA contained xylazine.” One can easily fathom the severity of this issue. Naloxone is commonly used as an antidote to many street drugs, but it is not effective against xylazine tainted fentanyl. Atipamezole and naloxone combination can be effective against xylazine tainted fentanyl. It can be also effective against many other drugs. It can be a life-saving antidote for several drug addicts. Thus, there is an urgent need to make combination drug therapy of naloxone and atipamezole. As the drug addicts can have different profiles, it was deemed necessary to develop various kinds of dosage forms. The compositions described herein can be understood by people with ordinary skills working in the field of drug formulation development and the compositions can be modified based on the needs of clinician's requirements. The excipients used in these compositions can be replaced with other commonly used excipients.

### REFERENCES

1. A. Pettersson; Non-abusable pharmaceutical composition comprising opioids; U.S. Pat. No. 8,470,361; Jun. 25, 2013.
2. A. Pettersson; Non-abusable pharmaceutical composition comprising opioids; U.S. Pat. No. 8,658,198; Feb. 25, 2014,
3. G. L. Myers, S. D. Hilbert, B. J. Boone, B. A. Bogue, P. Sanghvi, and M. Hariharan; Sublingual and buccal film compositions; U.S. Pat. No. 8,475,832; Jul. 2, 2013.
4. G. L. Myers, S. D. Hilbert, B. J. Boone, B. A. Bogue, P. Sanghvi, and M. Hariharan; Sublingual and buccal film compositions; U.S. Pat. No. 11,135,216; Oct. 5, 2021.

1. A method of treating drug addict consisting the steps of: a) providing a combination dosage form composition consisting: i) a therapeutically effective amount of naloxone or a pharmaceutically acceptable salt thereof; ii) a therapeutically effective amount of atipamezole or a pharmaceutically acceptable salt thereof; and b) administering said composition parenterally.

2. A dosage form in claim 1 further comprising a chelating agent.

3. A dosage form in claim 1 is administered intravenously.

4. A dosage form in claim 1 is administered intramuscularly.

5. A dosage form in claim 1 is administered subcutaneously.

6. A method of treating drug addict consisting the steps of: a) providing a combination drug dosage form composition consisting: i) a therapeutically effective amount of naloxone or a pharmaceutically acceptable salt thereof; ii) a therapeutically effective amount of atipamezole or a pharmaceutically acceptable salt thereof; and b) administering said composition orally.

7. A dosage form in claim 6 is an immediate release tablet or a capsule or a film.

8. A dosage form in claim 6 is a sublingual tablet.

9. A dosage form in claim 6 is an orally disintegrating tablet.

10. A dosage form in claim 6 is a sublingual orally disintegrating tablet.

11. A dosage form in claim 6 is an effervescent tablet.

12. A dosage form in claim 6 is a gelling tablet.

13. A dosage form in claim 6 is an orally dissolving film.

14. A method of treating a drug addict consisting the steps of: a) providing a combination dosage form composition consisting: i) a therapeutically effective amount of naloxone or a pharmaceutically acceptable salt thereof; ii) a therapeutically effective amount of atipamezole or a pharmaceutically acceptable salt thereof; and b) administering said composition topically.

15. A dosage form in claim 14 further comprising a chelating agent.

16. A dosage form in claim 14 is administered in the naso-pulmonary region.

17. A dosage form in claim 14 is administered topically on the skin.

18. A dosage form in claim 14 is a clear solution.

19. A dosage form in claim 14 is a gel.
